(19) United States
(12) Patent Application Publication
(10) Pub. No.: US 2014/0353188 A1
(43) Pub. Date: Dec. 4, 2014

(54) TRANSPORTATION INTEGRITY DEVICE FOR MEDICAL INSTRUMENTS

(71) Applicant: Covidien LP, Mansfield, MA (US)

(72) Inventors: ARLEN J. RESCHKE, LONGMONT, CO (US); ANTHONY D. RICKE, BOULDER, CO (US)

(73) Assignee: Covidien LP, Mansfield, MA (US)

(21) Appl. No.: 14/169,358

(22) Filed: Jan. 31, 2014

Related U.S. Application Data
(60) Provisional application No. 61/830,782, filed on Jun. 4, 2013.

(51) Int. Cl. A61B 19/02; B65D 77/02

(52) U.S. Cl. CPC A61B 19/026; B65D 77/02
USPC 206/363

(57) ABSTRACT

Packaging for a medical device includes a housing having an exterior surface and an interior surface including a compartment defined therein configured to contain a medical device. A carrier strip is secured to the exterior surface and/or the interior surface of the housing and includes one or more reactive portions disposed thereon. The reactive portion is configured to transition from a first visible state to a second visible state upon being subject to a defined parameter, temperature, radiation, sunlight, UV, microwave, etc.

### CROSS REFERENCE TO RELATED APPLICATION

The present application claims the benefit of and priority to U.S. Provisional Application Ser. No. 61/830,782, filed on Jun. 4, 2013, the entire contents of which are incorporated herein by reference.

### BACKGROUND

1. Technical Field

The present disclosure relates to carrier strips and sensors and, more particularly, to carrier strips or sensors configured to allow an end user to easily verify the integrity of a medical device that may have been subject to undesirably conditions during manufacture, handling and transport prior to opening the packaging thereof.

2. Background of Related Art

Medical devices are typically packaged in tub-like, peelable container or compartment that includes a thermoplastic film that is formed to a desired package shape and a lid material that is then sealed to the plastic film to contain the packaged medical product. Common thermoplastic film packages are often utilized to package individual medical devices or medical kits that are typically used for single use surgical procedures. The lid material used is commonly a non-woven fiber arranged such that it has a microporous structure, such as Tyvek®. The microporous fiber arrangement allows sterilization gas, e.g., ethylene oxide and steam sterilization, to penetrate but has a sufficiently small pore size to block the transfer of microorganisms. The Tyvek® lid does not allow the transmission of sunlight and ultraviolet light, however, the remainder of the external packaging may be directly exposed to sunlight and ultraviolet light for decontamination which may unnecessarily compromise the integrity of the medical instrument.

Moreover, during manufacturing, handing and transport, the medical instrument may become subject to other undesirable conditions that may compromise the instrument, affect the overall performance of the instrument, or may affect surgical outcome, e.g., sterilization integrity, radiation, and/or extreme temperatures (or temperatures outside a preferred range). Prior to opening the packaging or testing the instrument, it is very difficult to detect or determine if an instrument has been compromised or otherwise affected due to the instrument being subject to one or more of these undesirable conditions.

### SUMMARY

In accordance with aspects of the present disclosure, packaging for a medical device, includes a housing having an exterior surface and an interior surface. The interior surface includes a compartment defined therein configured to contain a medical device. A carrier strip is secured to the exterior and/or interior surfaces of the housing and includes one or more reactive portions disposed thereon. The reactive portion is configured to transition from a first visible state to a second visible state upon being subject to a defined parameter.

In one aspect of the present disclosure, the first and second visible states include color, texture, pattern, indicia, alphanumerics, and symbols. In another aspect, the defined parameter is selected from a group consisting of temperature, exposure to sunlight, exposure to UV, elapsed time, pressure, impact, exposure to a corrosive liquid or gas, exposure to radiation, exposure to ambient conditions, exposure to air, sterilization integrity, and exposure to microwave.

In still another aspect of the present disclosure, the carrier strip may be secured to the exterior surface and the defined parameter may include any one or more of transport time beyond a threshold condition, exposure to climate beyond at least one threshold condition, or impact above a threshold condition. In yet another aspect, the carrier strip is included in the interior surface of the housing and the defined parameter may include one or more of exposure to ambient conditions, exposure to radiation, exposure to temperatures above or below predetermined thresholds, and sterilization integrity.

In one aspect, the carrier strip may be secured to the housing by an adhesive, strap, mechanical coupling, magnetic coupling, or welding. The carrier strip may be secured to the exterior surface, be disposed or secured to the interior surface or both.

In another aspect, the carrier strip is disposed on a medical device and is configured to withstand autoclave sterilization without affecting the integrity of the one or more reactive portions and the defined parameter may include exposure to radiation and/or exposure to temperature below a predetermined threshold. In yet another aspect, the carrier strip is disposed on the medical device or on the interior surface of the housing and is configured to withstand gas sterilization without effecting the integrity of the one or more reactive portions and wherein the defined parameter may include any one of exposure to radiation, exposure to temperature above or below predetermined thresholds, exposure to ambient conditions, exposure to radiation or corrosive materials and sterilization integrity.

The present disclosure also relates to packaging for a medical device having a housing including an exterior surface and an interior surface, the interior surface including a compartment defined therein configured to contain a medical device. One or more sensors are secured to the exterior and/or interior surfaces of the housing. The sensor(s) is configured to provide feedback to a visual indicator upon being subject to a defined parameter or condition. The sensor(s) may be secured to the exterior surface of the housing, interior surface or both.

In one aspect, the sensor includes one or more of a bio-feedback sensor, electronic sensor, mechanical sensor, optical sensor, active sensor, passive sensor, and reactive sensor. The visual indicator may include one or more of color, texture, pattern, indicia, alphanumerics, and symbols. The defined parameter may include any one of temperature, change in temperature, exposure to sunlight, exposure to UV, elapsed time, pressure, impact, exposure to a corrosive liquid or gas, exposure to radiation, exposure to ambient conditions, exposure to air, sterilization integrity, exposure to microwave, etc.

In one aspect, the sensor(s) is secured to the exterior surface and the defined parameter includes any one or more of transport time beyond a threshold condition, exposure to climate beyond at least one threshold condition, or impact above a threshold condition. In another aspect, the sensor(s) is included in the interior surface of the housing or on the medical instrument and the defined parameter includes any one or more of exposure to ambient conditions, exposure to radiation, exposure to temperature above or below predetermined thresholds, and sterilization integrity.

In yet another aspect, the sensor(s) is secured to the housing by an adhesive, strap, mechanical coupling, magnetic coupling, or welding. In yet another aspect, the sensor is secured to the medical device and may be configured to withstand autoclave sterilization without affecting the integrity thereof and the defined parameter may include exposure to radiation and/or exposure to temperature below a predetermined threshold. The sensor may be configured to withstand gas sterilization without effecting the integrity of thereof and the defined parameter may include any one or more of exposure to radiation, exposure to temperature above or below predetermined thresholds, exposure to ambient conditions, and sterilization integrity.

### BRIEF DESCRIPTION OF THE DRAWINGS

Various aspects of the present disclosure are described herein with reference to the drawings wherein like reference numerals identify similar or identical elements:

FIG. 1 is a schematic, front, perspective view of packaging for a medical device employing a carrier strip with reactive portions thereon disposed on an exterior surface thereof;

FIG. 2 is a schematic, front, perspective view of packaging for a medical device employing a carrier strip with reactive portions thereon disposed on an interior surface thereof;

FIG. 3A is an enlarged, schematic view of the reactive portions of the carrier strip of FIGS. 1 and 2 shown in a first or neutral state;

FIG. 3B is an enlarged, schematic view of the reactive portions of the carrier strip of FIGS. 1 and 2 shown in a second or activated state with a pattern displayed thereon;

FIG. 4A is an enlarged, schematic view of the reactive portions of the carrier strip of FIGS. 1 and 2 shown in a first or neutral state;

FIG. 4B is an enlarged, schematic view of the reactive portions of the carrier strip of FIGS. 1 and 2 shown in a second or activated state with a symbol displayed thereon;

FIG. 5A is an enlarged, schematic view of the reactive portions of the carrier strip of FIGS. 1 and 2 shown in a first or neutral state showing a normal temperature condition;

FIG. 5B is an enlarged, schematic view of the reactive portions of the carrier strip of FIGS. 1 and 2 shown in a second or activated state showing that the packaging has been exposed to a high temperature condition;

FIG. 5C is an enlarged, schematic view of the reactive portions of the carrier strip of FIGS. 1 and 2 shown in a second or activated state showing that the packaging has been exposed to a low temperature condition;

FIG. 6A is a schematic, front, perspective view of packaging for a medical device employing a mechanical coupling for securing the carrier strip to the exterior or interior surface of the packaging;

FIG. 6B is an enlarged, schematic, perspective view of the carrier strip of FIG. 6A showing the an adhesive acting as the mechanical coupling for securing the carrier strip to the exterior surface of the packaging;

FIG. 7 is a schematic, front, perspective view of packaging for a medical device employing a sensor and visual indicator disposed on an exterior surface thereof;

FIG. 8A is an enlarged, schematic view of the visual indicator displaying feedback from the sensor relating to a non-sterile condition of the packaging;

FIG. 8B is an enlarged, schematic view of the visual indicator displaying feedback from the sensor relating indicating that the packaging was subject to an abnormal condition during manufacture, handling or transport; and

FIG. 8C is an enlarged, schematic view of the visual indicator displaying feedback from the sensor indicating that the package has been exposed to a hazardous or corrosive material during manufacture, handling or transport.

### DETAILED DESCRIPTION

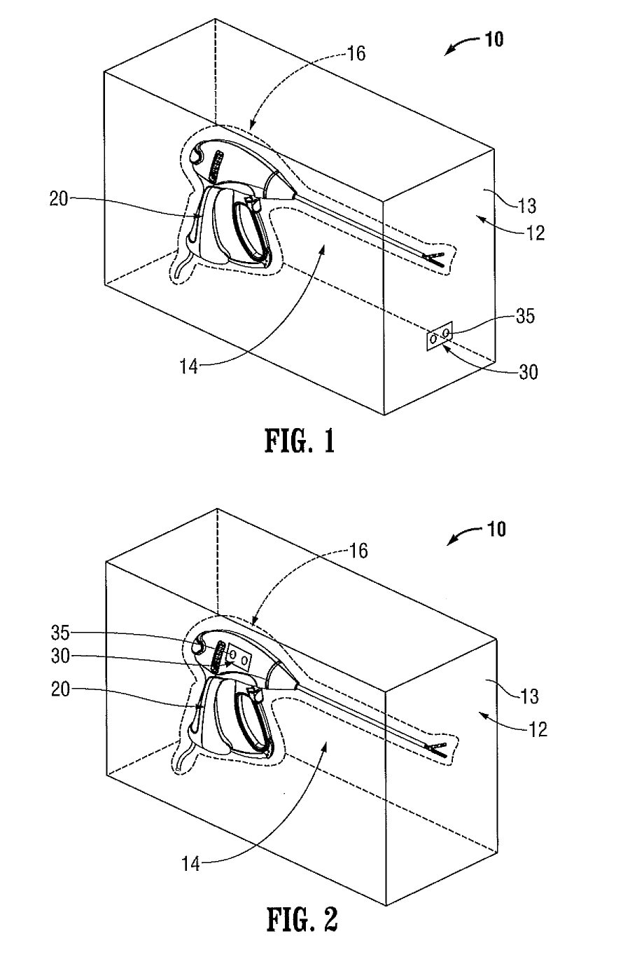

Referring initially to FIGS. 1 and 2, packaging for a medical instrument is provided in accordance with the present disclosure and is generally identified by reference numeral 10. Packaging includes a housing 12 having exterior and interior surfaces 13 and 14, respectively, that form a generally rectilinear or polygonal container for housing and securing a medical instrument 20. Typically, packaging 10 is made from clear plastic and includes a peelable film Tyvec® disposed thereon or at least includes a cover made from some other type of transparent material to allow a surgeon to verify the contents of the packaging prior to exposing the instrument 20 to the environment and compromising the instrument for use.

The interior surface 14 is configured to define a bin or compartment 16 (shown in phantom In FIGS. 1 and 2) shaped to secure the instrument 20 for handling and transport. Although the various figures show a forceps 20 for use with the present disclosure, any type of medical instrument 20 is envisioned that is normally packaged for handling and transport.

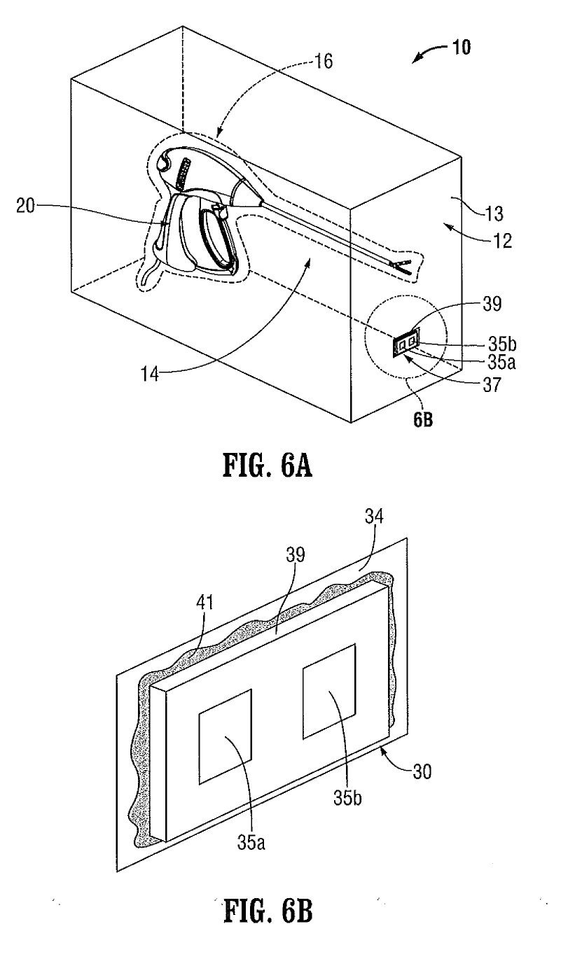

As best shown in FIG. 1, the exterior surface 13 of the packaging 10 includes a carrier strip 30 disposed thereon. Carrier strip 30 may be mechanically coupled to the exterior surface 13 in any known manner, e.g., mechanical coupling, strap, adhesive, magnetic coupling, welding, a synthetic material sold in ribbon, sheet, or piece goods form, the material having complemental parts which adhere to each other when pressed together and adapted for use as a closure fastener, or button for closing garments, curtains, or the like; separable fasteners-namely, hook and loop-type fasteners and components thereof commonly sold under the trademark VELCRO®, or the like. FIGS. 6A and 6B show the carrier strip 30 including a backing 39 that is secured to a predefined surface 34 of the housing 12 by an adhesive 41. As mentioned above, any mechanical method of securing the carrier strip 30 to the housing 12 may be utilized.

Carrier strip 30 (or an exposed portion thereof) includes one or more reactive portions 35 disposed thereon that are configured to transition from a first visible state to a second visible state upon being subject to a defined parameter. For example, the reactive portions 35 are transitionable from a first or normal visible state to a second or abnormal visible state if the package is subject to a defined parameter or condition outside the normal manufacturing conditions, handling conditions or transport conditions. These conditions or parameters include temperature, exposure to sunlight, exposure to UV, elapsed time, pressure, impact, exposure to a corrosive liquid or gas, exposure to radiation, exposure to ambient conditions, exposure to air, sterilization integrity, and exposure to microwave or other energy. In certain instances, the second visual state may be triggered (or otherwise become visible) upon the smallest amount of exposure to any one or more of the aforementioned parameters, e.g., radiation, sterile environment, etc.

Alternatively (or, in combination with two or more reactive portions 35 being utilized), the second state transition may be triggered (or otherwise become visibly apparent) if one or more of the above defined parameters falls outside a normal operating range or above or below a threshold condition, e.g., high or low temperature, excess exposure to sunlight, excess exposure to UV, excess transport or handling time, excess pressure on the packaging 10, abnormal wear and tear or impact, excess exposure to a corrosive liquid or gas, excess exposure to radiation, excess exposure to ambient conditions, excess exposure to air, and/or excess exposure to microwave waves or other harmful energy.

As best show in FIG. 2, the carrier strip 30 is disposed on the interior surface 14 of the housing 12. As mentioned above with respect to FIG. 1, the carrier strip 30 may be affixed to the packaging 10 in known mechanical fashion or in this case with the carrier strip 30 being disposed within the packaging 10, the carrier strip 30 may be simply disposed within a cavity or depression (not shown) within the packaging 10, on the instrument 20 or otherwise simply placed therein. Much like above with respect to the carrier strip of FIG. 1, the reactive portions 35 of the carrier strip 30 are configured to visible change when subject to a particular defined parameter, upon the carrier strip 30 being subject to a condition outside a given range or the carrier strip being subjected to a threshold condition, e.g., temperature, exposure to sunlight, exposure to UV, elapsed time, pressure, impact, exposure to a corrosive liquid or gas, exposure to radiation, exposure to ambient conditions, exposure to air, sterilization integrity, and exposure to microwave or other energy.

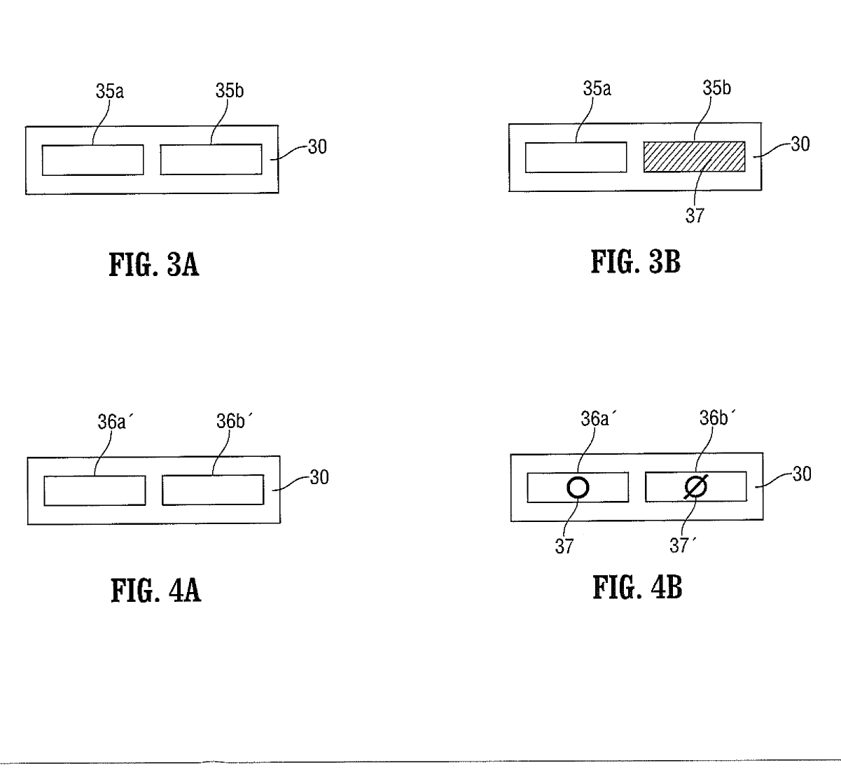

FIGS. 3A-5C illustrate examples of carrier strips 30 and reactive portions 35 wherein the reactive portions transition from a first visible state to a second visible state. More particularly, FIGS. 3A-3B show the transition of reactive portion 35b from a solid surface 35b in FIG. 3A to a textured or patterned surface 37 on portion 35b in FIG. 3B. Reactive portion 35a remains unchanged (not exposed to a different parameter). Changes to the first and second visible states may, include color changes, texture changes, pattern changes, indicia or other markings, alphanumerics markings, and/or symbols. For example, FIGS. 4A-4B show a different type of visible state reaction wherein reactive portion 36a′ and 36b′, after transport or handing, the include symbols representing safe handling and transport “O” (reference numeral 37) for one parameter and exposure “Ø” (reference numeral 37′) to a predetermined parameter for another. Any symbol 37 may be employed for this purpose.

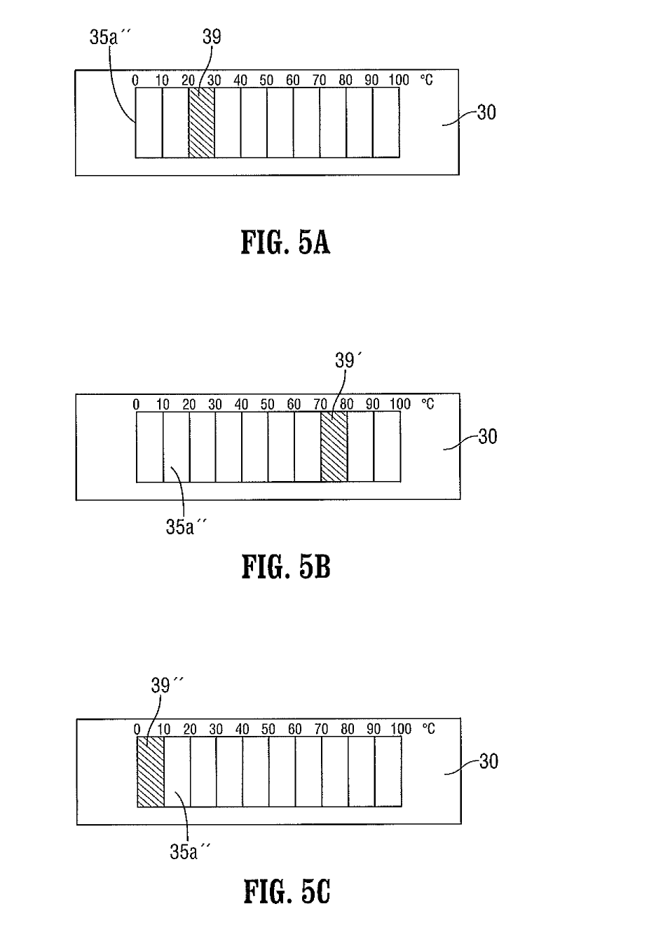

In addition, the reactive portions 35a″ may be able to provide a graphical information that shows a degree of exposure to a certain parameter, e.g., temperature, energy, radiation, sunlight, etc. as best shown in FIG. 5A-5C. FIG. 5A shows temperature within a normal range as identified by marker 39 for ideal transport conditions, FIG. 5B indicating that the packaging 10 or carrier strip 30 has been subject to high temperature conditions as identified by marker 39′ and FIG. 5C indicating that the packaging and carrier strip 30 has been subject to low temperature conditions as identified by marker 39″. Any type of marker or indicia may be utilized for this purpose and the markers 39, 39′ and 39″ may be more or less sophisticated depending upon a particular purpose, e.g., multiple temperature markers, time and temperature markers (how long the carrier strip 30 was exposed to an undesirable temperature condition), and/or a simple color-code marker (blue—too cold, red—too hot).

In one embodiment, the carrier strip 30 is attached to the medical instrument 20 and is configured to withstand autoclave sterilization without affecting the integrity of the one or more reactive portions 35 and wherein the defined parameter includes exposure to or excess radiation, sunlight, UV, energy, etc. or exposure to temperature below a predetermined threshold (due to high temperatures of autoclave sterilization). When gas sterilization is utilized, the carrier strip 30 may be attached to the instrument 20 or disposed within the interior surface 14 and configured to withstand as sterilization without affecting the integrity of the reactive portion(s) 35 and the defined parameters may include any of the aforementioned and, particularly, exposure to or excess radiation, sunlight, UV, temperature above or below predetermined thresholds, ambient conditions, and sterilization integrity. In either instance of gas sterilization, the carrier strip 30 may be disposed on the exterior surface 13 or interior surface 14 of the housing 12 depending upon a particular purpose.

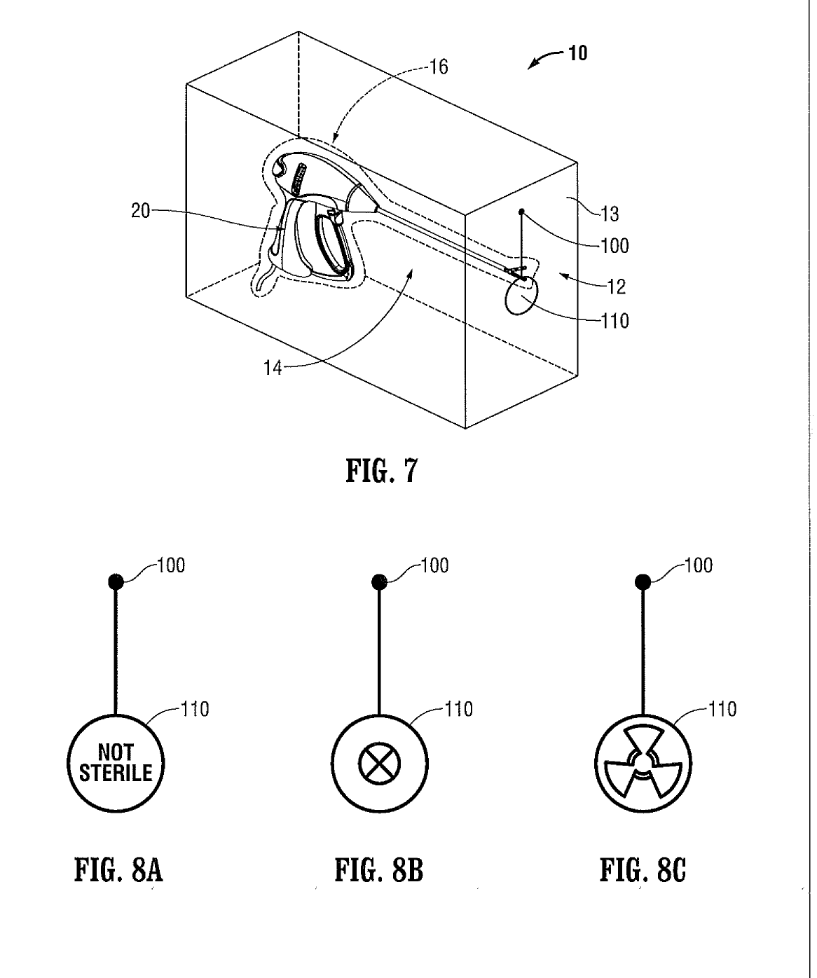

As shown in FIGS. 7-8C, the present disclosure also relates to packaging 10 for a medical device that includes a housing 12 having an exterior surface 13 and an interior surface 14 with a compartment 16 (shown in phantom) defined therein configured to contain a medical device 20. One or more sensors 100 are secured to the exterior and/or interior surfaces, 13, 14 of the housing 12 and are configured to provide feedback to a visual indicator 110 upon being subject to a defined parameter. Many different types of sensors 100 are envisioned and include bio-feedback sensors, electronic sensors, mechanical sensors, optical sensors, active sensors, passive sensors, and reactive sensors. The visual indicator 110 may be configured to provide information to the end user relating to undesirable or abnormal conditions concerning manufacture, handling, or transport. This information can be displayed in virtually any envisioned format, e.g., color, texture, pattern, indicia, alphanumerics, and/or symbols.

As mentioned above, the defined parameters may include exposure to abnormal temperatures, changes in temperature, exposure to or excess sunlight, exposure to or excess UV, excess transport time, exposure to or excess pressure on the packaging 10, impact (package drop or crush), exposure to a corrosive liquid or gas, exposure to or excess radiation, exposure to ambient conditions, exposure to air, sterilization integrity, and exposure to microwave or other types of energy that may affect the integrity of the medical instrument 20. Multiple types of feedback may be conveyed to the visual indicator 110 and the end user may be able to access a menu or graphical user interface (GUI) prior to breaking the packaging 10 seal.

As can be appreciated, manufacturers can track shipments utilizing one or more of the aforementioned visual indicators 110 and carrier strips 30 and may be able to control manufacturing, handling and transport conditions to insure a more reliable product is delivered to the end user. End users, on the other hand, will be able to get visual confirmation prior to opening the packaging for the medical instrument 20, e.g., that the medical instrument 20 was not compromised in any fashion (as defined by the particular parameters set by the manufacturer during manufacture, handling or transport due to conditions beyond the manufacturer's control). If for some reason the instrument 20 was subject to one or more undesirable conditions falling outside the manufacturer's recommended parameter range, the user may simply return the instrument 20 to the manufacturer and utilize a different instrument 20 during surgery.

This may prove particularly useful with respect to sterilization integrity of the instrument 20 or exposure of the entire packaging 10 to hazardous materials, e.g., radiation. For example, the visual indicator 110 may display “Not Sterile” (See FIG. 8A) if the integrity of the medical instrument is compromised or a radiation symbol may appear if the packaging 10 has been exposed to radiation (See FIG. 8C). As can be appreciated, any symbol may be utilized to visually indicate to the user that the instrument 20 or packaging 10 has been subject to an undesirable parameter or condition during manufacture, handling and transport (See FIG. 8B).

The sensor 100 may be disposed on the interior surface of the housing 12 and attached to the instrument 20 and configured to withstand autoclave or gas sterilization without affecting the integrity of the sensor 100 or the visual indicator 110. If utilizing autoclave sterilization, the defined parameters may include exposure to or excess radiation, sunlight, UV, energy, etc. or exposure to temperature below a predetermined threshold (due to high temperatures of autoclave sterilization). When gas sterilization is utilized, the sensor 100 may be attached to the instrument 20 or disposed within the interior surface 14 and configured to withstand gas sterilization without effecting the integrity of the sensor 100 and the defined parameters may include any of the aforementioned and, particularly, exposure to or excess radiation, sunlight, UV, temperature above or below predetermined thresholds, ambient conditions, and sterilization integrity. In one instance, the sensor 100 may be disposed on the interior surface 14 of the housing 12 and capable of withstanding gas sterilization and the visual indicator 110 is disposed on the exterior surface 13 of the housing 12.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed:

1. Packaging for a medical device, comprising:
a housing including an exterior surface and an interior surface, the interior surface having a compartment defined therein configured to contain a medical device; and
a carrier strip secured to one of the exterior and interior surfaces of the housing, the carrier strip including at least one reactive portion disposed thereon, wherein the reactive portion transitions from a first visible state to a second visible state upon being subject to a defined parameter.

2. Packaging for a medical device according to claim 1 wherein the first and second visible states include one of color, texture, pattern, indicia, alphanumerics, and symbols.

3. Packaging for a medical device according to claim 1 wherein the defined parameter is selected from a group consisting of temperature, exposure to sunlight, exposure to UV, elapsed time, pressure, impact, exposure to a corrosive liquid or gas, exposure to radiation, exposure to ambient conditions, exposure to air, sterilization integrity, and exposure to microwave.

4. Packaging for a medical device according to claim 1 wherein the carrier strip is secured to the exterior surface and the defined parameter includes transport time beyond a threshold condition, exposure to climate beyond at least one threshold condition, or impact above a threshold condition.

5. Packaging for a medical device according to claim 1 wherein the carrier strip is included in the interior surface of the housing and the defined parameter includes exposure to ambient conditions, exposure to radiation, exposure to temperature above or below predetermined thresholds, and sterilization integrity.

6. Packaging for a medical device according to claim 1 wherein the carrier strip is secured to the housing by an adhesive, strap, mechanical coupling, magnetic coupling, or welding.

7. Packaging for a medical device according to claim 1 wherein the carrier strip is secured to the exterior surface of the housing.

8. Packaging for a medical device according to claim 1 wherein the carrier strip is disposed on medical device and is configured to withstand autoclave sterilization without effecting the integrity of the at least one reactive portion and wherein the defined parameter includes exposure to radiation and exposure to temperature below a predetermined threshold.

9. Packaging for a medical device according to claim 1 wherein the carrier strip is disposed on one of the medical device and the interior surface of the housing and is configured to withstand gas sterilization without effecting the integrity of the at least one reactive portion and wherein the defined parameter includes exposure to radiation, exposure to temperature above or below predetermined thresholds, exposure to ambient conditions, and sterilization integrity.

10. Packaging for a medical device, comprising:
a housing including an exterior surface and an interior surface, the interior surface having a compartment defined therein configured to contain a medical device; and
at least one sensor secured to one of the exterior and interior surfaces of the housing, the at least one sensor configured to provide feedback to a visual indicator upon being subject to a defined parameter.

11. Packaging for a medical device according to claim 10 wherein the sensor includes one of a bio-feedback sensor, electronic sensor, mechanical sensor, optical sensor, active sensor, passive sensor, and reactive sensor.

12. Packaging for a medical device according to claim 10 wherein the visual indicator includes one of color, texture, pattern, indicia, alphanumerics, and symbols.

13. Packaging for a medical device according to claim 10 wherein the defined parameter is selected from a group consisting of temperature, change in temperature, exposure to sunlight, exposure to UV, elapsed time, pressure, impact, exposure to a corrosive liquid or gas, exposure to radiation, exposure to ambient conditions, exposure to air, sterilization integrity, and exposure to microwave.

14. Packaging for a medical device according to claim 10 wherein the at least one sensor is secured to the exterior surface and the defined parameter includes transport time beyond a threshold condition, exposure to climate beyond at least one threshold condition, or impact above a threshold condition.

15. Packaging for a medical device according to claim 10 wherein the at least one sensor is included in the interior surface of the housing and the defined parameter includes exposure to ambient conditions, exposure to radiation, exposure to temperature above or below predetermined thresholds, and sterilization integrity.

16. Packaging for a medical device according to claim 10 wherein the at least one sensor is secured to the housing by an adhesive, strap, mechanical coupling, magnetic coupling, or welding.

17. Packaging for a medical device according to claim 10 wherein the at least one sensor is secured to the exterior surface of the housing.

18. Packaging for a medical device according to claim 10 wherein the at least one sensor is disposed on the medical device and is configured to withstand autoclave sterilization without effecting the integrity thereof and wherein the defined parameter includes exposure to radiation and exposure to temperature below a predetermined threshold.

19. Packaging for a medical device according to claim 10 wherein the at least one sensor is disposed on one of the medical device and the interior surface of the housing and is configured to withstand gas sterilization without effecting the integrity of thereof and wherein the defined parameter includes exposure to radiation, exposure to temperature above or below predetermined thresholds, exposure to ambient conditions, and sterilization integrity.
